(19) United States
(12) Patent Application Publication
(10) Pub. No.: US 2021/0291017 A1
(43) Pub. Date: Sep. 23, 2021

(54) EXERCISE ASSISTING APPARATUS, METHOD OF ASSISTING EXERCISE, AND NON-TRANSITORY RECORDING MEDIUM

(71) Applicant: CASIO COMPUTER CO., LTD., Shibuya-ku (JP)

(72) Inventor: Hiroki TOMITA, Suginami-ku (JP)

(73) Assignee: CASIO COMPUTER CO., LTD., Shibuya-ku (JP)

(21) Appl. No.: 17/164,944

(22) Filed: Feb. 2, 2021

(30) Foreign Application Priority Data
Mar. 19, 2020 (JP) 2020-048697

(51) Int. Cl. A63B 24/00; A63B 71/06

(52) U.S. Cl. CPC A63B 24/0006; A63B 71/0622; A63B 2220/44; A63B 2220/836; A63B 2071/0694

(57) ABSTRACT

An exercise assisting apparatus includes at least one memory and at least one processor configured to execute a program loaded in the memory. The processor resamples arm-swing trajectory data pieces on a plurality of human subjects with a predetermined number of samples. The processor generates a distance matrix on the basis of the minimum distance between two point groups after association between individual points, the two point groups being selected from among the resampled arm-swing trajectory data pieces. The processor generates clustering data through classification of the values contained in the distance matrix into a certain number of clusters.

### CROSS-REFERENCE TO RELATED APPLICATION

This application claims the benefit of Japanese Patent Application No. 2020-048697, filed on Mar. 19, 2020, the entire disclosure of which is incorporated by reference herein.

### FIELD

This application relates to an exercise assisting apparatus, a method of assisting exercise, and a non-transitory recording medium.

### BACKGROUND

A way of swinging arms is important during running and walking. The walking or running people, however, cannot readily recognize their own ways of swinging arms without paying careful attention. In particular, people who are tired tend to pay less attention to the ways of swinging arms. In view of this problem, for example, Unexamined Japanese Patent Application Publication No. 2019-115665, which is a patent literature filed with the Japan Patent Office, discloses a technique of estimating the amount of steps made by a user on the basis of the detected arm swing peaks of the user and calculating an energy consumption value.

### SUMMARY

An exercise assisting apparatus according to a preferred aspect of the disclosure includes at least one memory and at least one processor configured to execute a program loaded in the memory. The processor resamples arm-swing trajectory data pieces on a plurality of human subjects with a predetermined number of samples. The processor generates a distance matrix on the basis of the minimum distance between two point groups after association between individual points, the two point groups being selected from among the resampled arm-swing trajectory data pieces. The processor generates clustering data through classification of the values contained in the distance matrix into a certain number of clusters.

### BRIEF DESCRIPTION OF THE DRAWINGS

A more complete understanding of this application can be obtained when the following detailed description is considered in conjunction with the following drawings, in which:

FIG. 1 illustrates an exemplary procedure of acquiring arm-swing trajectory data pieces according to an embodiment of the disclosure;

FIG. 2 is a block diagram illustrating a configuration of an exercise assisting system according to the embodiment of the disclosure;

FIG. 3 illustrates a configuration of an exercise assisting apparatus according to the embodiment of the disclosure;

FIG. 4A illustrates a representative trajectory when an arm of a user swinging leftward in the traveling direction is viewed directly from the left of the user, in an exemplary result of clustering of arm-swing trajectories according to the embodiment of the disclosure;

FIG. 4B illustrates a representative trajectory when the arm of the user swinging frontward in the traveling direction is viewed directly from the front of the user, in an exemplary result of clustering of arm-swing trajectories according to the embodiment of the disclosure;

FIG. 4C illustrates a representative trajectory when the arm of the user swinging leftward in the traveling direction is viewed directly from above the user, in an exemplary result of clustering of arm-swing trajectories according to the embodiment of the disclosure;

FIG. 4D illustrates a representative trajectory when the arm of the user swinging rearward in the traveling direction is viewed directly from the back of the user, in an exemplary result of clustering of arm-swing trajectories according to the embodiment of the disclosure;

FIG. 5A illustrates a representative trajectory when the arm of the user swinging leftward in the traveling direction is viewed directly from the left of the user, in an exemplary result of clustering of arm-swing trajectories according to the embodiment of the disclosure;

FIG. 5B illustrates a representative trajectory when the arm of the user swinging frontward in the traveling direction is viewed directly from the front of the user, in an exemplary result of clustering of arm-swing trajectories according to the embodiment of the disclosure;

FIG. 5C illustrates a representative trajectory when the arm of the user swinging leftward in the traveling direction is viewed directly from above the user, in an exemplary result of clustering of arm-swing trajectories according to the embodiment of the disclosure;

FIG. 5D illustrates a representative trajectory when the arm of the user swinging rearward in the traveling direction is viewed directly from the back of the user, in an exemplary result of clustering of arm-swing trajectories according to the embodiment of the disclosure;

FIG. 6A illustrates a representative trajectory when the arm of the user swinging leftward in the traveling direction is viewed directly from the left of the user, in an exemplary result of clustering of arm-swing trajectories according to the embodiment of the disclosure;

FIG. 6B illustrates a representative trajectory when the arm of the user swinging frontward in the traveling direction is viewed directly from the front of the user, in an exemplary result of clustering of arm-swing trajectories according to the embodiment of the disclosure;

FIG. 6C illustrates a representative trajectory when the arm of the user swinging leftward in the traveling direction is viewed directly from above the user, in an exemplary result of clustering of arm-swing trajectories according to the embodiment of the disclosure;

FIG. 6D illustrates a representative trajectory when the arm of the user swinging rearward in the traveling direction is viewed directly from the back of the user, in an exemplary result of clustering of arm-swing trajectories according to the embodiment of the disclosure;

FIG. 7 illustrates an exemplary hardware configuration of the exercise assisting apparatus according to the embodiment of the disclosure;

FIG. 8 is a flowchart illustrating a trajectory clustering process executed in the exercise assisting apparatus according to the embodiment of the disclosure; and

FIG. 9 is a flowchart illustrating a trajectory type determining process executed in the exercise assisting apparatus according to the embodiment of the disclosure.

### DETAILED DESCRIPTION

An embodiment of the disclosure will now be described in detail with reference to the accompanying drawings. In these drawings, the components identical or corresponding to each other are provided with the same reference symbol.

An exercise assisting apparatus 2 according to an embodiment of the disclosure is able to cluster arm-swing trajectory data pieces acquired from a plurality of human subjects by a terminal 1 and thereby generate clustering data, and able to determine the arm-swing type of a user on the basis of the similar cluster identified through comparison between an arm-swing trajectory data piece on the user currently acquired by the terminal 1 and the generated clustering data.

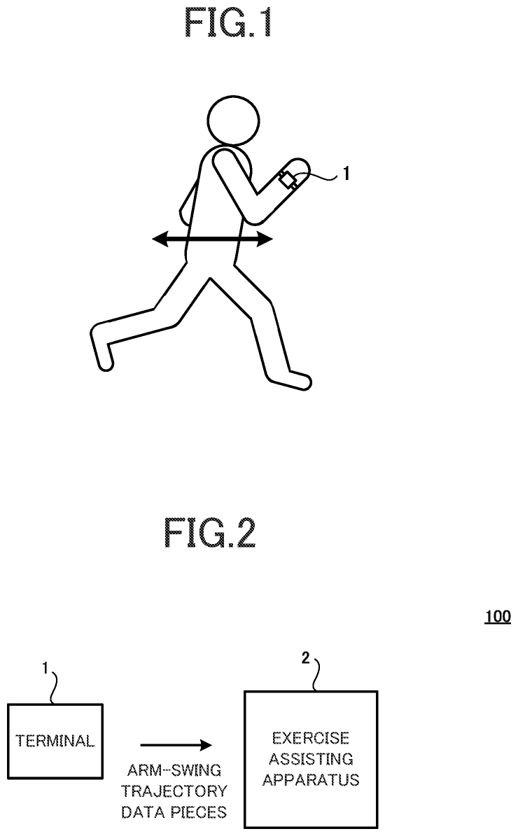

FIG. 1 illustrates a user equipped with the terminal 1 around an arm. The terminal 1 includes a sensor, such as acceleration sensor, gyro sensor, or geomagnetic sensor, and can acquire an arm-swing trajectory data piece on the user. The arm-swing trajectory data piece on the user indicates trajectory data on a single back-and-forth arm swing. The trajectory data on a single back-and-forth arm swing is, for example, data from when the arm starts to move frontward from the rear turnaround point until when the arm arrives at the original rear turnaround point via the front turnaround point. Alternatively, the trajectory data on a single back-and-forth arm swing may be data from when the arm starts to move rearward from the front turnaround point until when the arm arrives at the original front turnaround point via the rear turnaround point. The terminal 1 acquired the data measured by the sensor, such as acceleration sensor, gyro sensor, or geomagnetic sensor, in constant periods having a predetermined length, thereby acquiring trajectory data on a single back-and-forth arm swing. The trajectory data on multiple back-and-forth arm swings is acquired as explained above, which is hereinafter referred to as “arm-swing trajectory data pieces A”. The arm-swing trajectory data pieces A are point groups in the three dimensions of xyz and can be represented in Expression (1) below, for example. In Expression (1), n, m, and k each indicate an integer of at least 3.

$$\begin{matrix}[ Math 1 ] & \\ A = [ \begin{matrix}{a}_{x 1} , {a}_{x 2} , … , {a}_{xn} \\ {a}_{y 1} , {a}_{y 2} , … , {a}_{yn} \\ {a}_{z 1} , {a}_{z 2} , … , {a}_{zn}\end{matrix} ] , [ \begin{matrix}{a}_{x 1} , {a}_{x 2} , … , {a}_{xm} \\ {a}_{y 1} , {a}_{y 2} , … , {a}_{ym} \\ {a}_{z 1} , {a}_{z 2} , … , {a}_{zm}\end{matrix} ] , … , [ \begin{matrix}{a}_{x 1} , {a}_{x 2} , … , {a}_{xk} \\ {a}_{y 1} , {a}_{y 2} , … , {a}_{yk} \\ {a}_{z 1} , {a}_{z 2} , … , {a}_{zk}\end{matrix} ] & ( 1 )\end{matrix}$$

The arm-swing trajectory data pieces A generated in the terminal 1 are transmitted from the terminal 1 to the exercise assisting apparatus 2, as illustrated in FIG. 2. The exercise assisting apparatus 2 clusters the arm-swing trajectory data pieces A acquired from the terminal 1, depending on their shapes, and thereby generates clustering data. The exercise assisting apparatus 2 then compares arm-swing trajectory data pieces A currently acquired from the terminal 1 with the generated clustering data and thereby identifies a similar cluster. On the basis of the identified similar cluster, the exercise assisting apparatus 2 determines the arm-swing type of the user. The terminal 1 and the exercise assisting apparatus 2 are hereinafter collectively referred to as “exercise assisting system 100”.

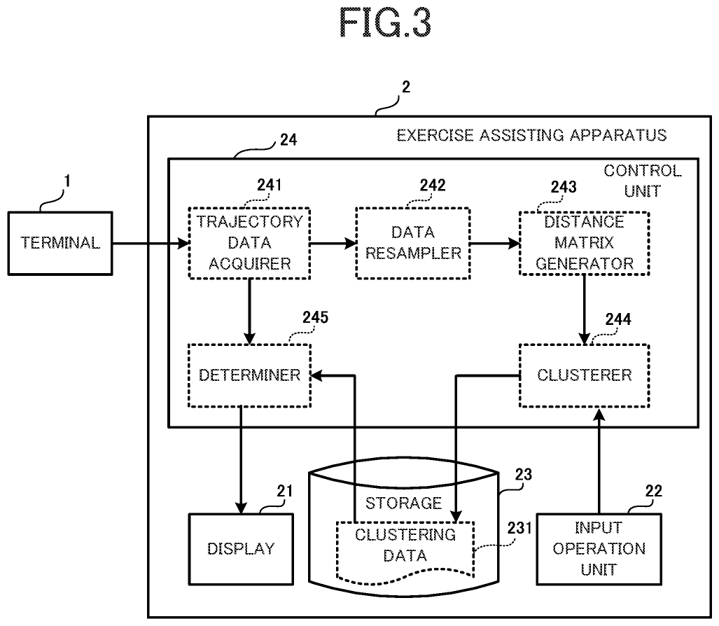

With reference to FIG. 3, the exercise assisting apparatus 2 includes a display 21 for displaying various types of data, an input operation unit 22 for receiving an instruction from a user, a storage 23 for storing various types of data and programs, and a control unit 24. The display 21 displays various types of data in accordance with an instruction from the control unit 24. The input operation unit 22 functions as an input unit for receiving an input from the user. The storage 23 stores clustering data 231, resulting from clustering of the arm-swing trajectory data pieces A depending on their shapes, various programs, data to be used in the programs, and other data.

The control unit 24 includes a trajectory data acquirer 241 for acquiring data from the terminal 1, a data resampler 242 for resampling the acquired data, a distance matrix generator 243 for generating a distance matrix from the resampled data, a clusterer 244 for clustering the values contained in the distance matrix, and a determiner 245 for determining the arm-swing type of the user in currently-acquired data.

The trajectory data acquirer 241 acquires arm-swing trajectory data pieces A from the terminal 1. The data resampler 242 fixes the positions of the initial and terminal points of each of the acquired arm-swing trajectory data pieces A and resamples the data between the initial and terminal points with a predetermined number of samples. The original data pieces have mutually different numbers of samples because of mutually different periods required for a single back-and-forth arm swing. The process of resampling with the predetermined number of samples can equalize the number of samples in all the trajectories of back-and-forth arm swings. The resampled arm-swing trajectory data pieces are hereinafter referred to as “arm-swing trajectory data pieces B”. In an exemplary case of resampling with 50 samples, the arm-swing trajectory data pieces B resampled from the individual arm-swing trajectory data pieces A are represented as arm-swing trajectory data pieces B1 to BN in Expression (2) below. In Expression (2), N indicates an integer of at least 2.

$$\begin{matrix}[ Math 2 ] & \\ B 1 = [ \begin{matrix}b {1}_{x 1} , b {1}_{x 2} , … , b {1}_{x 50} \\ b {1}_{y 1} , b {1}_{y 2} , … , b {1}_{y 50} \\ b {1}_{z 1} , b {1}_{z 2} , … , b {1}_{z 50}\end{matrix} ] \\ ⋮ \\ BN = [ \begin{matrix}{bN}_{x 1} , {bN}_{x 2} , … , {bN}_{x 50} \\ {bN}_{y 1} , {bN}_{y 2} , … , {bN}_{y 50} \\ {bN}_{z 1} , {bN}_{z 2} , … , {bN}_{z 50}\end{matrix} ] & ( 2 )\end{matrix}$$

The distance matrix generator 243 generates a distance matrix C from the arm-swing trajectory data pieces B1 to BN represented in Expression (2). Since the numbers of samples in the individual arm-swing trajectory data pieces A are all equalized, the respective points from the initial to terminal points of two trajectories can be associated with each other. The two point groups after association between the individual points are subject to translation, rotation, or uniform scaling conversion, and then superimposed on each other so as to provide the minimum squared error between the two point groups, that is, the minimum distance between the two point groups. This process can yield the Procrustes distance between these two point groups. The Procrustes distance is a scalar value. The Procrustes distance is calculated from each of the arm-swing trajectory data pieces B1 to BN. The calculated Procrustes distances are combined into a single matrix, which is the distance matrix C. In an exemplary case of 100 arm-swing trajectory data pieces containing the arm-swing trajectory data pieces B1 to B100, the resulting matrix is a 100×100 matrix. The distance matrix C in this case is represented in Expression (3) below, for example.

$$\begin{matrix}[ Math 3 ] & \\ C = \vert \begin{matrix}0 , {c}_{12} , {c}_{13} , & … & , {c}_{1 \; 100} \\ {c}_{21} , 0 , {c}_{23} , & … & , {c}_{2 \; 100} \\ ⋮ & ⋱ & ⋮ \\ {c}_{100 \; 1 ,} {c}_{100 \; 2} , {c}_{100 \; 3} , & … & 0\end{matrix} \vert & ( 3 )\end{matrix}$$

The clusterer 244 classifies the values contained in the generated distance matrix C into any number D of clusters. Examples of clustering algorithm include unsupervised clustering algorithms, such as k-means and k-medoids. The k-means algorithm involves acquiring the midpoint in each cluster every clustering process. The k-medoids algorithm involves selecting, as the centroid (center) of the cluster, the data piece having the minimum total distance from the other data pieces in each cluster. In this embodiment, the values contained in the distance matrix C are classified into any number D of clusters by the k-medoids algorithm, for example.

For each of the D clusters resulting from classification, the clusterer 244 automatically or manually determines the shape of the representative trajectory as viewed in each direction of xyz and provides a meaning. To provide a meaning indicates to provide a name of type suggested by the shape and characteristics of the representative trajectory as viewed in each direction of xyz. The name of type is “rotary type”, “8-shaped type”, and “L-shaped type”, for example. The meaning is automatically provided by the exercise assisting apparatus 2, with reference to the shapes as viewed in each direction of xyz and the names of type stored in the storage 23. Alternatively, the user may observe the representative trajectory as viewed in each direction of xyz displayed on the display 21 and input a name of type suggested by the shape and characteristics of the trajectory through the input operation unit 22. The clusterer 244 causes the D clusters provided with meanings to be stored into the storage 23 in the form of the clustering data 231.

The determiner 245 compares an arm-swing trajectory data piece E on a single back-and-forth swing, which is currently acquired by the trajectory data acquirer 241 from the terminal 1, with the clustering data 231 stored in the storage 23, and thereby determines the arm-swing type. The determiner 245 causes the result of determination to be displayed on the display 21. Specifically, the determiner 245 acquires an arm-swing trajectory data piece E on a single back-and-forth swing from the arm-swing trajectory data pieces currently acquired by the trajectory data acquirer 241 from the terminal 1. The determiner 245 then acquires the D clusters from the clustering data 231 stored in the storage 23. The determiner 245 then calculates Procrustes distances between the arm-swing trajectory data piece E on a single back-and-forth swing and the individual D clusters, and thus identifies the cluster having the minimum Procrustes distance, that is, the similar cluster. The determiner 245 then causes the name of the identified cluster having the minimum Procrustes distance to be displayed on the display 21. The name of the cluster on the display 21 is the name of the arm-swing type.

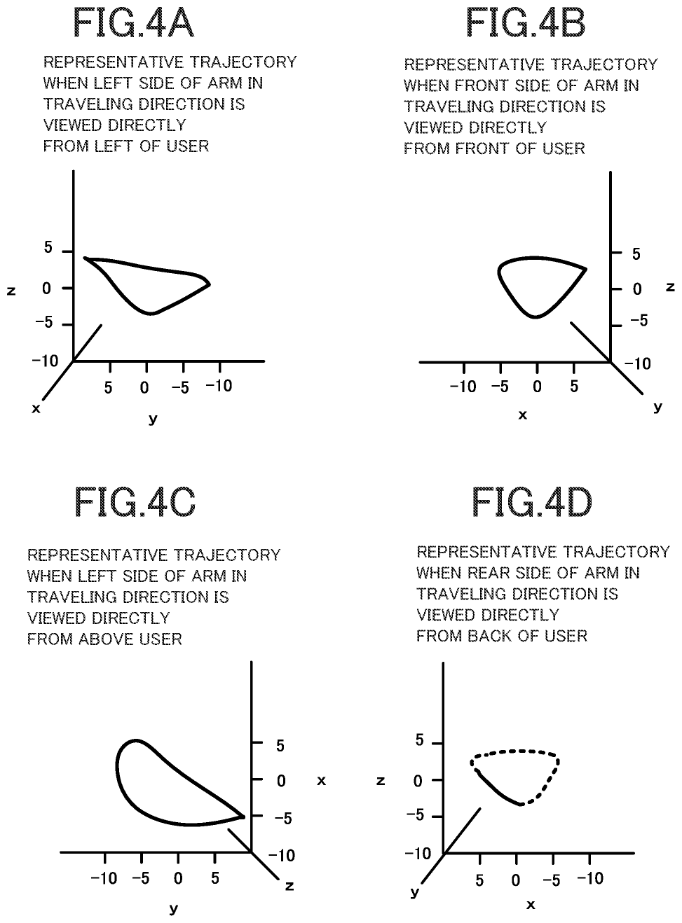

Examples of the D clusters resulting from classification in the clusterer 244 and the meanings provided to the respective clusters are illustrated in FIGS. 4 to 6. FIGS. 4A, 5A, and 6A each illustrate a representative trajectory when an arm of a user swinging leftward in the traveling direction is viewed directly from the left of the user. FIGS. 4B, 5B, and 6B each illustrate a representative trajectory when the arm of the user swinging frontward in the traveling direction is viewed directly from the front of the user. FIGS. 4C, 5C, and 6C each illustrate a representative trajectory when the arm of the user swinging leftward in the traveling direction is viewed directly from above the user. FIGS. 4D, 5D, and 6D each illustrate a representative trajectory when the arm of the user swinging rearward in the traveling direction is viewed directly from the back of the user. Each of the representative trajectories represents a shape as viewed in each direction of xyz. It should be noted that the dashed line in each of FIGS. 4D, 5D, and 6D indicates the segment other than the segments contained in the representative trajectories illustrated in FIGS. 4A to 4C, 5A to 5C, and 6A to 6C.

The representative trajectories illustrated in FIGS. 4A to 4C each have a ring shape having the overlapped initial and terminal points. The representative trajectory illustrated in FIG. 4D has a ring shape having the overlapped initial and terminal points if the dashed-line segment is also taken into consideration as well as the solid-line segment. Such a ring-shaped trajectory seems to be derived from a ring-shaped movement, that is, rotation of the arm. This trajectory is therefore named as rotary type, for example. In this embodiment, the representative trajectories illustrated in FIGS. 4A to 4D are named as rotary type.

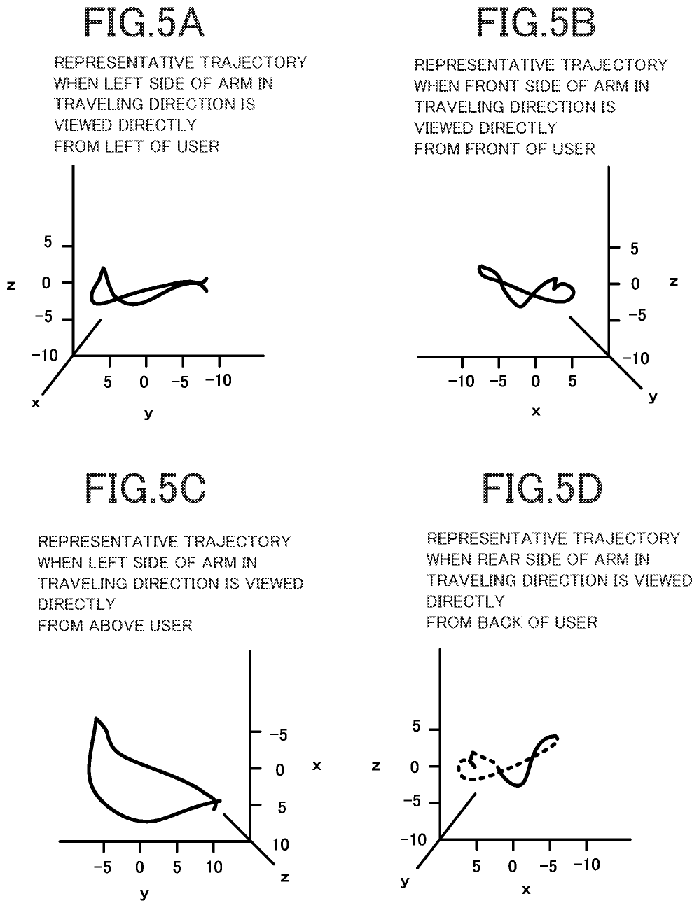

The representative trajectory illustrated in FIG. 5A has a shape like an alphabet “L”. This trajectory is therefore named as L-shaped type. The representative trajectory illustrated in FIG. 5B contains mutually intersecting segments and has a shape like a figure “8”. The representative trajectory illustrated in FIG. 5D contains mutually intersecting segments and has a shape like a figure “8” if the dashed-line segment is also taken into consideration as well as the solid-line segment. The representative trajectories illustrated in FIGS. 5B and 5D are therefore named as 8-shaped type. The representative trajectory illustrated in FIG. 5C has a ring shape having the substantially overlapped initial and terminal points. The representative trajectory illustrated in FIG. 5C is therefore named as rotary type.

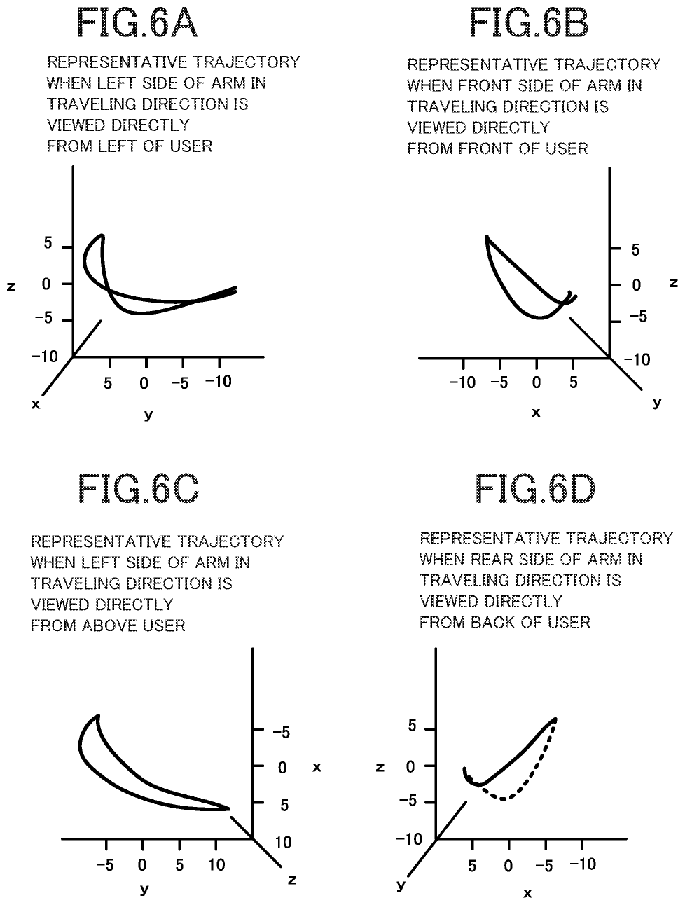

The representative trajectory illustrated in FIG. 6A has a shape like an alphabet “L”. This trajectory is therefore named as L-shaped type. The representative trajectory illustrated in FIG. 6B represents a sharp motion from the initial point to the terminal point. The representative trajectory illustrated in FIG. 6D represents a sharp motion from the initial point to the terminal point if the dashed-line segment is also taken into consideration as well as the solid-line segment. The representative trajectories illustrated in FIGS. 6B and 6D are therefore named as sharp motion type. The representative trajectory illustrated in FIG. 6C has two segments having substantially linear shapes between the initial and terminal points. This trajectory is therefore named as linear type.

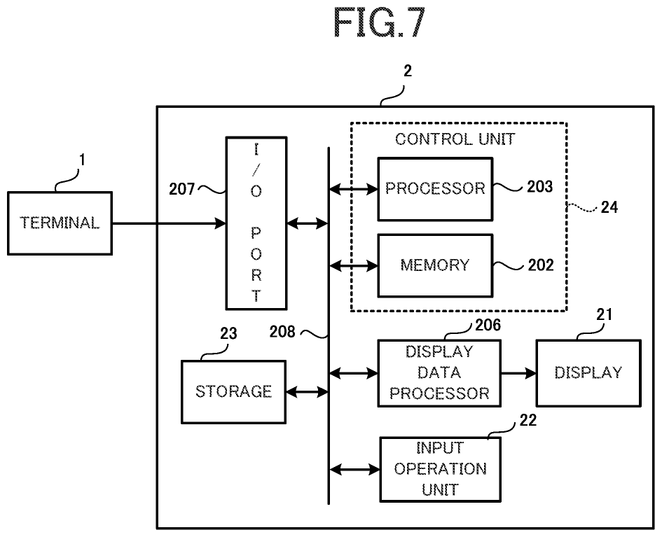

The clusterer 244 illustrated in FIG. 3 provides meanings to a predetermined number of clusters, for example, six clusters among the resulting D clusters, in the descending order of the number of data pieces belonging to the cluster. The trajectory data acquirer 241, the data resampler 242, the distance matrix generator 243, the clusterer 244, and the determiner 245 in the control unit 24 illustrated in FIG. 3 are functions achieved by execution of programs stored in the storage 23. An exemplary hardware configuration of the exercise assisting apparatus 2 for executing these programs will now be described with reference to FIG. 7.

The exercise assisting apparatus 2 is configured by electronic equipment, such as a server apparatus or personal computer. The exercise assisting apparatus 2 is equipped with the storage 23 for storing various programs and various types of data, the control unit 24 including at least one memory 202 in which various programs are loaded and at least one processor 203 for executing the various programs loaded in the memory 202, the input operation unit 22 for receiving an input from the user, the display 21 for displaying various types of data, a display data processor 206 for generating and outputting display data to be displayed on the display 21, an I/O port 207 capable of establishing connection to the terminal 1, and internal buses 208. The storage 23, the memory 202, the processor 203, the input operation unit 22, the display 21, the display data processor 206, and the I/O port 207 are connected to each other via the internal buses 208.

The storage 23 stores various programs for achieving various functions performed in the exercise assisting apparatus 2, and various types of data containing the clustering data 231 illustrated in FIG. 3. The storage 23 includes, for example, a read-only memory (ROM) or a memory element. The memory 202 is a memory element in which various programs read from the storage 23 can be loaded. The memory 202 includes, for example, a random access memory (RAM).

The processor 203 executes various programs loaded in the memory 202. The processor 203 includes a control element, such as central processing unit (CPU) or micro-processing unit (MPU), for example. The trajectory data acquirer 241, the data resampler 242, the distance matrix generator 243, the clusterer 244, and the determiner 245 in the control unit 24 illustrated in FIG. 3 are achieved by execution of programs stored in the storage 23 by the processor 203. The input operation unit 22 is an input unit for receiving instructions and inputs of various character strings from the user. The input operation unit 22 includes, for example, a keyboard, key buttons, a tablet, or a digitizer.

The display 21 displays various types of data. The display 21 includes, for example, an organic electroluminescence (EL) display, a liquid crystal display (LCD), or a display panel capable of maximum intensity projection (MIP) display. The display data processor 206 generates various types of display data and outputs the data to the display 21. The display data processor 206 includes an image signal output device, such as video card, graphics processing unit (GPU), or graphics board, for example. The I/O port 207 can establish connection to the terminal 1. The I/O port 207 is any of various ports capable of establishing connection to devices, such as universal serial bus (USB) port or IEEE 1394 port, for example.

A trajectory clustering process and a trajectory type determining process executed in the exercise assisting apparatus 2 will now be explained. The trajectory clustering process involves clustering the arm-swing trajectory data pieces on the user acquired from the terminal 1 and generating D clusters. The trajectory type determining process involves comparing an arm-swing trajectory data piece on a single back-and-forth swing of a user currently acquired from the terminal 1 with the clusters generated in the trajectory clustering process and determining the arm-swing type of the user.

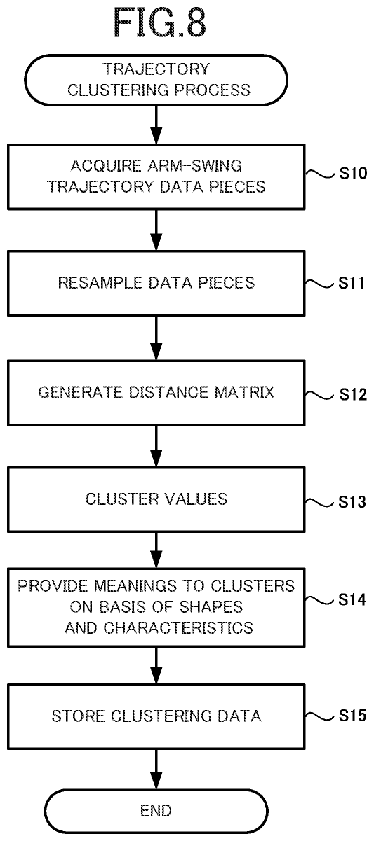

The trajectory clustering process is stored in the storage 23 illustrated in FIG. 7 in the form of a trajectory clustering program. In response to an instruction from a user, for example, selection of an object, such as icon or menu, displayed on the display 21, the exercise assisting apparatus 2 loads the trajectory clustering program in the memory 202. The trajectory clustering program loaded in the memory 202 is executed by the processor 203. The trajectory clustering process will now be explained with reference to the flowchart illustrated in FIG. 8.

The trajectory data acquirer 241 of the exercise assisting apparatus 2 illustrated in FIG. 3 acquires arm-swing trajectory data pieces, which are arm-swing trajectory data pieces on multiple back-and-fourth swings of a user, from the terminal 1 (Step S10). The arm-swing trajectory data pieces are assumed to be the arm-swing trajectory data pieces A represented in the above Expression (1), for example. The data resampler 242 of the exercise assisting apparatus 2 resamples each of the arm-swing trajectory data pieces A with the predetermined number of samples (Step S11). In an exemplary case of resampling with 50 samples, the arm-swing trajectory data pieces B resampled from the individual arm-swing trajectory data pieces A are represented as arm-swing trajectory data pieces B1 to BN in above Expression (2).

The distance matrix generator 243 of the exercise assisting apparatus 2 illustrated in FIG. 3 then generates a distance matrix from the arm-swing trajectory data pieces B (Step S12). In an exemplary case of 100 arm-swing trajectory data pieces containing the arm-swing trajectory data pieces B1 to B100, the resulting matrix is a 100×100 matrix. The distance matrix C in this case is represented in above Expression (3), for example.

The clusterer 244 of the exercise assisting apparatus 2 clusters the values contained in the generated distance matrix C (Step S13). Specifically, the clusterer 244 clusters the values contained in the generated distance matrix C into any number D of clusters. The clusterer 244 provides meanings to the clusters on the basis of the shapes and characteristics of the representative trajectories belonging to the respective clusters (Step S14). For example, the clusters are named as rotary type, 8-shaped type, or L-shaped type, on the basis of the shapes and characteristics of the representative trajectories, as illustrated in FIGS. 4 to 6. The clusterer 244 causes the D clusters provided with meanings to be stored into the storage 23 in the form of the clustering data 231 (Step S15).

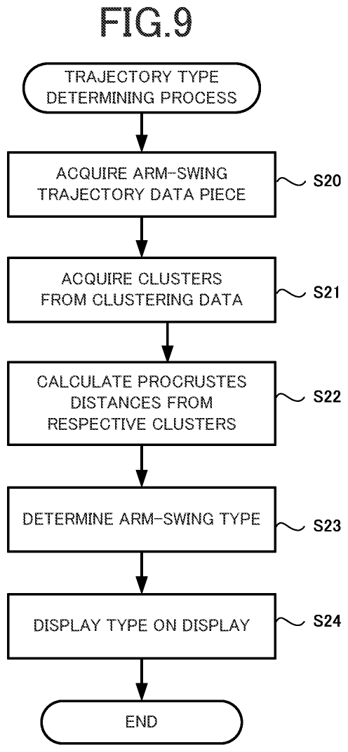

The trajectory type determining process is stored in the storage 23 illustrated in FIG. 7 in the form of a trajectory type determining program. In response to an instruction from a user, for example, selection of an object, such as icon or menu, displayed on the display 21, the exercise assisting apparatus 2 loads the trajectory type determining program in the memory 202. The trajectory type determining program loaded in the memory 202 is executed by the processor 203. The trajectory type determining process will now be explained with reference to the flowchart illustrated in FIG. 9.

The determiner 245 of the exercise assisting apparatus 2 illustrated in FIG. 3 acquires an arm-swing trajectory data piece E on a single back-and-forth swing from the arm-swing trajectory data pieces currently acquired by the trajectory data acquirer 241 from the terminal 1 (Step S20). The determiner 245 then acquires the D clusters provided with meanings from the clustering data 231 stored in the storage 23 (Step S21).

The determiner 245 calculates Procrustes distances between the arm-swing trajectory data piece E on a single back-and-forth swing and the respective D clusters (Step S22). The determiner 245 then determines the cluster having the minimum Procrustes distance as the arm-swing type (Step S23). The determiner 245 causes the name of the determined cluster having the minimum Procrustes distance to be displayed on the display 21 (Step S24).

As described above, the exercise assisting apparatus 2 according to the embodiment is able to cluster arm-swing trajectory data pieces acquired from a plurality of human subjects and thereby generate clustering data. The exercise assisting apparatus 2 can thus determine the arm-swing type of a user.

The exercise assisting apparatus 2 according to the embodiment may also detect the stability of arm swings, and a variation in arm swings with time due to a temperature change in the place where the user is running or a degree of fatigue of the user, on the basis of the consistency of the arm-swing type of the user or a variation in the arm-swing type with time, for example.

(Modification)

The above-described embodiment should not be construed as limiting the disclosure and may be provided with various modifications without departing from the gist of the disclosure.

In the above-described embodiment, the arm-swing trajectory data pieces A are generated on the basis of data acquired by the sensor, such as acceleration sensor or gyro sensor, included in the terminal 1. Alternatively, the arm-swing trajectory data pieces A may be generated on the basis of three-dimensional segment data obtained by estimating the attitude of the user from the data on marker coordinates in a motion capture system. It should be noted that the terminal 1 and the motion capture system are each an exemplary external device in the claims.

In this embodiment, the clustering data is generated from the arm-swing trajectory data pieces acquired from a plurality of human subjects by the terminal 1. Alternatively, the clustering data may be generated from arm-swing trajectory data pieces on a simulation model or a famous track-and-field athlete, for example.

In this embodiment, the exercise assisting apparatus 2 is configured as an apparatus that is able to cluster arm-swing trajectory data pieces acquired from a plurality of human subjects by the terminal 1 and thereby generate clustering data, and able to determine the arm-swing type on the basis of the similar cluster identified through comparison between an arm-swing trajectory data piece on the user currently acquired by the terminal 1 and the generated clustering data. Alternatively, an exercise assisting system may be configured by a clustering data generating apparatus for clustering arm-swing trajectory data pieces acquired from a plurality of human subjects by the terminal 1 and thereby generating clustering data, and an arm-swing type determining apparatus for storing the clustering data generated in the clustering data generating apparatus and determining the arm-swing type of a user on the basis of the similar cluster identified through comparison between an arm-swing trajectory data piece on the user currently acquired by the terminal 1 and the stored clustering data.

The trajectory clustering program and the trajectory type determining program in this embodiment may be applied in any procedure. For example, the trajectory clustering program and the trajectory type determining program may be applied in the form of being stored in a non-transitory computer-readable recording medium, such as flexible disk, compact disc (CD)-ROM, digital versatile disc (DVD)-ROM, or memory card. In addition, a display control program may be superimposed on a carrier wave and applied via a communication medium, such as the Internet. For example, the program may be posted and distributed via a bulletin board system (BBS) on a communication network. A processor may execute the above-explained processes by activating and executing the display control program under the control of an operating system (OS) as well as other application programs.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

What is claimed is:

1. An exercise assisting apparatus comprising:
at least one memory; and
at least one processor configured to execute a program loaded in the memory, wherein
the processor
resamples arm-swing trajectory data pieces on a plurality of human subjects with a predetermined number of samples,
generates a distance matrix on basis of a minimum distance between two point groups after association between individual points, the two point groups being selected from among the resampled arm-swing trajectory data pieces, and
generates clustering data through classification of values contained in the distance matrix into a certain number of clusters.

2. The exercise assisting apparatus according to claim 1, wherein the processor provides a name to each of the clusters resulting from classification.

3. The exercise assisting apparatus according to claim 1, wherein the processor compares a currently-acquired arm-swing trajectory data piece on a user with the clustering data, identifies a similar cluster that is similar to the currently-acquired arm-swing trajectory data piece and corresponds to the clustering data, and determines an arm-swing type on basis of the identified similar cluster.

4. The exercise assisting apparatus according to claim 3, wherein the processor causes the determined arm-swing type to be displayed on a display.

5. The exercise assisting apparatus according to claim 1, wherein the processor provides a name to each of the clusters resulting from classification, the name being input by a user.

6. The exercise assisting apparatus according to claim 1, wherein the arm-swing trajectory data pieces are data acquired by a terminal comprising a sensor.

7. A method of assisting exercise executed in an exercise assisting apparatus, the method comprising:
resampling arm-swing trajectory data pieces on a plurality of human subjects with a predetermined number of samples;
generating a distance matrix on basis of a minimum distance between two point groups after association between individual points, the two point groups being selected from among the resampled arm-swing trajectory data pieces; and
generating clustering data through classification of values contained in the distance matrix into a certain number of clusters.

8. A non-transitory recording medium storing a program thereon, the program causing a computer to:
resample arm-swing trajectory data pieces on a plurality of human subjects with a predetermined number of samples;
generate a distance matrix on basis of a minimum distance between two point groups after association between individual points, the two point groups being selected from among the resampled arm-swing trajectory data pieces; and
generate clustering data through classification of values contained in the distance matrix into a certain number of clusters.
